(19) United States
(12) Patent Application Publication
(10) Pub. No.: US 2009/0012576 A1
(43) Pub. Date: Jan. 8, 2009

(54) CONNECTOR ASSEMBLY FOR IMPLANTABLE MEDICAL DEVICE

(75) Inventors: Blair Erbstoeszer, Kirkland, WA (US); Mark G. Deehr, Woodinville, WA (US); Richard W. Swenson, Edina, MN (US); Mee S. Burckhardt, Redmond, WA (US)

Correspondence Address: SCHWEGMAN, LUNDBERG & WOESSNER, P.A., P.O. BOX 2938, MINNEAPOLIS, MN, 55402, US

(73) Assignee: Cardiac Pacemakers, Inc.

(21) Appl. No.: 12/143,721

(22) Filed: Jun. 20, 2008

Related U.S. Application Data
(60) Provisional application No. 60/936,431, filed on Jun. 20, 2007.

(51) Int. Cl. A61N 1/02

(52) U.S. Cl. 607/38

(57) ABSTRACT

Header assemblies for coupling one or more cardiac leads to a cardiac stimulator are described. The header assemblies can include a header housing with a lead receiving header bore. The header assemblies can further include a connector assembly injection molded within the header housing and including a connector housing, at least one biasing member and at least one biasing member cover. The connector housing can include a first connector bore aligned with the header bore and at least a second connector bore. The biasing member can be in the form of a coiled spring or bent wire and disposed within the second connector bore such that a portion of the biasing member projects into the first connector bore. In this way, a lead proximal end can be urged against a first connector bore wall. The biasing member cover can secure the biasing member and seal the second connector bore.

### CLAIM OF PRIORITY

This non-provisional application claims the benefit of priority under 35 U.S.C. § 119(e) to U.S. Provisional Patent Application Ser. No. 60/936,431, filed on Jun. 20, 2007, which is herein incorporated by reference.

### TECHNICAL FIELD

This patent document pertains generally to implantable medical devices. More particularly, but not by way of limitation, this patent document pertains to a connector assembly for connecting one or more cardiac leads to a cardiac stimulator, such as a pacemaker or defibrillator.

### BACKGROUND

The course of treatment indicated for patients suffering from a cardiac arrhythmia normally depends on a number of factors, such as the age of a patient, the type and severity of the arrhythmia, as well as other factors. Some patients may be successfully treated using drug therapy, surgical intervention, or a combination of the two. However, for other patients, a course of treatment may involve direct electrical stimulation of the heart's affected area by means of an implanted cardiac stimulator.

Implantable cardiac stimulator systems can consist of a cardiac stimulator and one or more elongated cardiac leads. The cardiac stimulator may be a pacemaker, a defibrillator, a sensing instrument, or some combination thereof. The circuitry, batteries, and other components of the cardiac stimulator are ordinarily encased within a metallic housing, which is commonly referred to as a “can”. Most of the circuitry of the cardiac stimulator is mounted on an electronic circuit board commonly known as a multi-chip module or hybrid microcircuit.

A proximal end of a cardiac lead of the cardiac stimulator system can be connected physically and electrically to the cardiac stimulator can via a structure commonly known as a header. A distal end of the cardiac lead can be implanted near the site requiring electrical stimulation or sensing. The cardiac lead functions to carry electrical stimulation signals from the cardiac stimulator can to the targeted tissue or to transmit sensing signals from the targeted tissue back to the cardiac stimulator can.

In a common procedure used by physicians to implant a new cardiac stimulator system, a cardiac lead is first implanted inside a patient's body and manipulated so that the distal end of the cardiac lead is positioned proximate the targeted heart tissue. The proximal end of the cardiac lead is normally left protruding from the body during the implantation procedure so that it may be readily connected to the cardiac stimulator. After the distal end of the cardiac lead has been positioned inside the body as desired, the proximal end of the cardiac lead is connected to the header by inserting it into a relative large lead receiving bore and tightening a header set-screw.

One or more conductor wires emanating from the cardiac stimulator can may be coupled to one or more electrical contacts provided inside the header. The one or more electrical contacts inside the header may be tubular in shape or provided with tubular passages and are typically fabricated with inner diameters that are large relative to an outer diameter of the proximal end of the cardiac lead, thereby providing sliding fits between the contacts and the proximal end. A sliding fit has conventionally been preferable for enabling the implanting physician to insert the proximal end of the cardiac lead with minimal effort and with little risk of damaging the lead or the header. Following connection of each of the system's cardiac leads, the cardiac stimulator can be implanted under the patient's skin.

### SUMMARY

The present inventors have recognized, among other things, that as a result of the relatively loose fit between the one or more electrical contacts and the proximal end of a cardiac lead, the lead may make only intermittent electrical contact or no contact at all with the header until the set-screw is tightened. As a result, reliable electrical conduction between the lead and the electrical contacts is not ensured until the set-screw is tightened leading to possible time delay between the moment when the proximal end of the cardiac lead is inserted into the header and when the set-screw is tightened by the physician. Unfortunately, some arrhythmia patients may be adversely impacted by even short interruptions in the application of electrical stimulus to the heart, even in circumstances where the surgeon has made efforts to minimize the disconnection time.

The present inventors have further recognized that it may be beneficial to have at least a second means of lead retainment with the header beyond a set-screw. Many modern cardiac stimulators have an anticipated implant life span of five years or longer. Following implantation, the connection between the cardiac stimulator and a cardiac lead is subjected to a variety of stresses that stem from the patient's physical activity or the rhythmic motion of the patient's breathing and heart beat. Some patients even place stress on the header/cardiac lead connection by habitually palpating their implanted cardiac stimulators with their hands. In addition, high stresses may be imparted by physical trauma to the body. Years of exposure to such stresses may loosen the set-screw. It is believed that in the absence of at least a secondary engaging mechanism, the lead may disconnect from the header.

In light of the foregoing and other inventor recognitions, header assemblies for coupling one or more cardiac leads to a cardiac stimulator are described in this document. The header assemblies can include a header housing with a header bore configured to receive a proximal end of a cardiac lead. The header assemblies can further include a connector assembly injection molded within the header housing and including a connector housing, at least one biasing member and at least one biasing member cover. The connector housing can include a first connector bore aligned with the header bore and at least a second connector bore disposed at an angle to the header bore. The biasing member can provide for secondary retainment of the cardiac in the header assembly and can be in the form of a biasing spring or bent wire, for example. When disposed within the second connector bore, the biasing member acts to urge the lead proximal end against a wall of the first connector bore. The biasing member cover can secure the biasing member within the connector housing and inhibit material flow into the second connector bore during injection molding of the header housing, thus inhibiting the formation of unwanted flash.

The injection molding of the connector assembly within the header housing may advantageously provide for lower cardiac stimulator manufacturing costs, more robust securement of the connector assembly within the header housing and more repeatable alignment between a header bore and a first connector bore, as the connector assembly does not have to be press-fit into the header housing after the header housing has been molded. Further, the present header assemblies include a biasing member held within a second connector bore in such a way that the biasing member interferes with the first connector bore, which is configured to receive the proximal end of a cardiac lead. In this way, consistent and immediate electrical contact between the cardiac lead and the connector assembly is possible.

These and other examples, advantages, and features of the present header assemblies and methods will be set forth in part in following Detailed Description. This Summary is intended to provide an overview of the subject matter of the present patent document. It is not intended to provide an exclusive or exhaustive explanation of the present subject matter. The Detailed Description is included to provide further information about the present patent document.

### BRIEF DESCRIPTION OF THE DRAWINGS

In the drawings, like numerals have been used to describe similar components throughout the several views. Like numerals having different letter suffixes have been used to represent different instances of similar components. The drawings illustrate generally, by way of example, but not by way of limitation, various embodiments described in the present document.

FIG. 1 illustrates an example of a schematic view of a cardiac stimulator system including a cardiac stimulator and a cardiac lead.

FIG. 2 illustrates an example of a cross-sectional view of a header assembly taken at line 2-2 of FIG. 1 and a proximal end of a cardiac lead.

FIG. 3 illustrates an example of an exploded schematic view of a connector assembly and a proximal end of a cardiac lead.

FIG. 4 illustrates an example of an isometric view of a bent wire biasing member.

FIG. 5 illustrates an example of an interference arrangement between a first connector bore and a coiled spring biasing member disposed in a second connector bore.

FIG. 6 illustrates another example of an interference arrangement between a first connector bore and a bent wire biasing member disposed in a second connector bore.

FIG. 7 illustrates an example of an unexploded schematic view of a connector assembly and a proximal end of a cardiac lead.

FIG. 8 illustrates an example of an exploded schematic view of a connector assembly and a proximal end of a cardiac lead.

FIGS. 9A-9F illustrate example orientation views of a connector housing, including at least first and second connector bores.

FIGS. 10A-10F illustrate example orientation views of a connector housing, including at least first and second connector bores.

FIG. 11 illustrates an example of an isometric view of a connector assembly and a proximal end of a cardiac lead.

FIG. 12 illustrates an example of a schematic view of a cardiac stimulator system including a cardiac stimulator and two cardiac leads.

### DETAILED DESCRIPTION

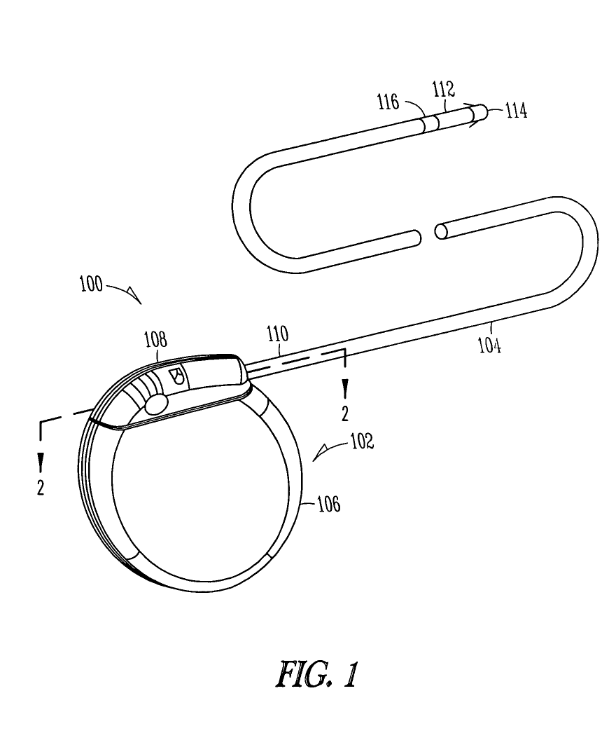

Turning to the drawings, and in particular to FIG. 1, there is shown an example of an implantable cardiac stimulator system 100 that can be suitable for either endocardial or epicardial stimulation of a human heart. The cardiac stimulator system 100 includes a cardiac stimulator 102 and one or more cardiac leads 104. Each lead 104 can be of such length that it is shown broken. The cardiac stimulator 102 can consist of a housing (can) 106, composed of titanium or other metallic material, connected to a header assembly 108. The can 106 may be configured to encase the electronic components of the cardiac stimulator 102, which may include storage cells, power transistors, microprocessors, telemetry circuits, sensors, or induction coils for rechargeable storage cells, among others. It should be understood that the term “cardiac stimulator” may refer to a pacemaker, a defibrillator, a sensing instrument, or some combination of these devices.

As shown, a proximal end 110 of the lead 104 can be connected to the header assembly 108. A distal end 112 of the lead 104 can terminate in a tip electrode 114, for example, that is designed to be attached to or disposed near the tissue warranting electrical stimulation. In this example, the lead 104 is shown in a bipolar configuration. Accordingly, the lead 104 is provided with a second electrode 116, which can be located proximal to the tip electrode 114. Beyond bipolar lead configurations, unipolar, quadpolar and other lead configurations can also be connected to the header assembly 108.

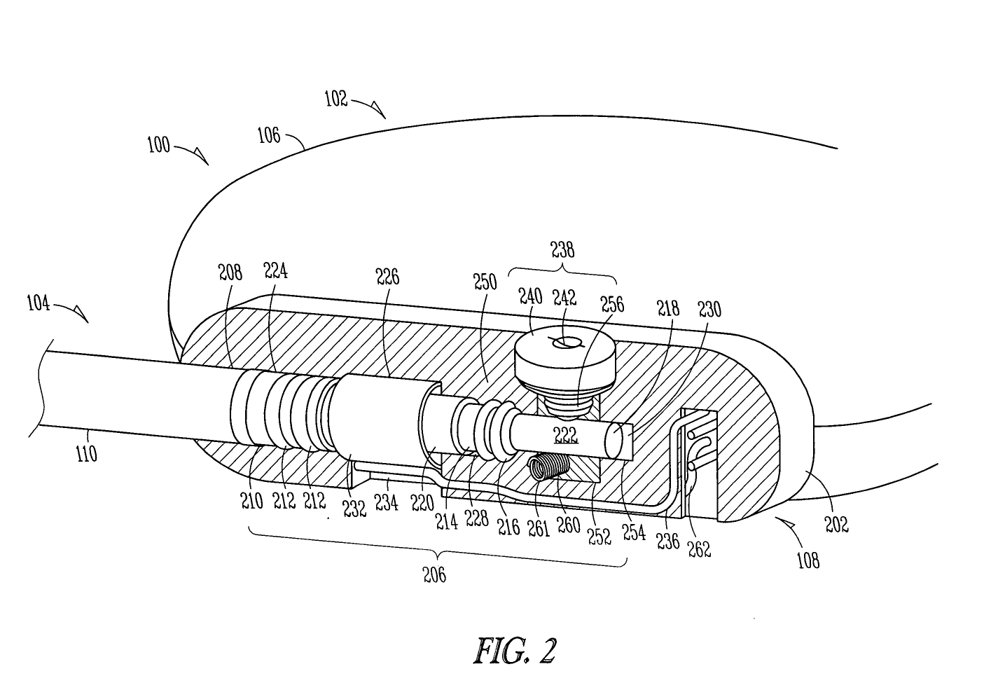

The detailed structure of the header assembly 108 and the connection thereof with the lead 104 may be further understood by referring to FIG. 2, which illustrates a cross-sectional view of FIG. 1 taken along line 2-2. The header assembly 108 can include a header housing 202 composed of epoxy, molded plastic or like materials. The proximal end 110 of the lead 104 can include a connector 206 that is disposed within a longitudinal header bore 208 in the header housing 202. The connector 206 can have three segments, including a distal segment 210 that is provided with O-rings 212, an intermediate segment 214 that is provided with O-rings 216, and a proximal segment 218. The connector intermediate segment 214 can include one or more terminal ring contacts 220 that are in electrical communication with an electrode, such as electrode 116 (FIG. 1), via a conductor wire disposed inside the lead 104. The connector proximal segment 218 can include a terminal pin contact 222 in electrical communication with another electrode, such as the tip electrode 114 (FIG. 1), via a conductor wire running inside the lead 104.

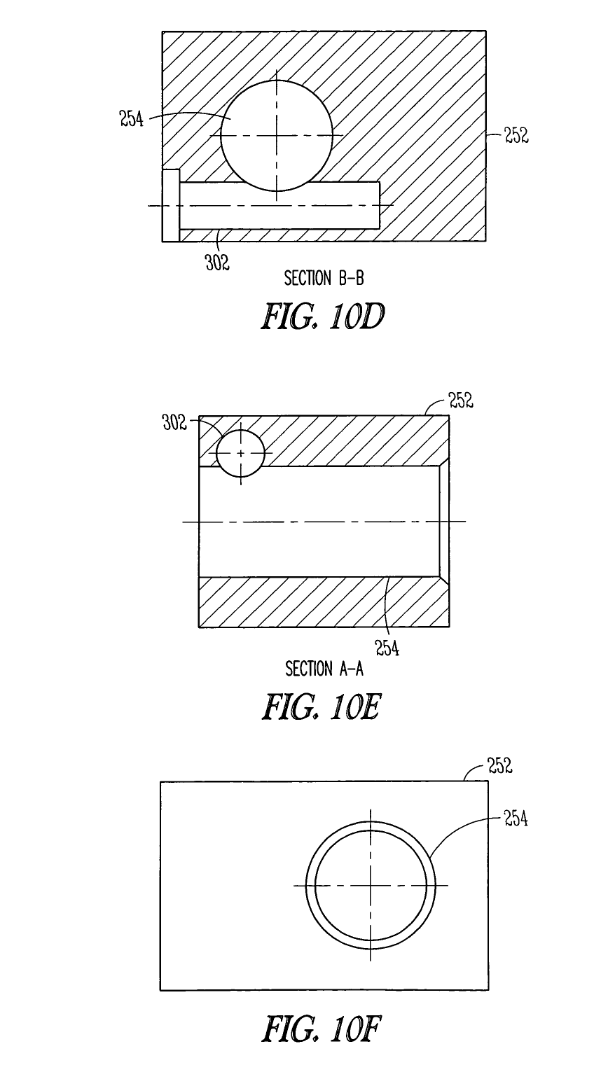

The header bore 208 can include four sections, a distal section 224, a first intermediate section 226, a second intermediate section 228, and a proximal section 230. The header bore distal section 224 can be sized to accommodate the connector distal segment 210 and to provide sealing engagement with the O-rings 212 to restrict the influx of body fluids that could impede electrical performance of the cardiac stimulator system 100. The proximal section 230 of the header bore 208 can be sized to axially receive the proximal segment 218 of the connector assembly 206. The second header bore intermediate section 228 can be sized to accommodate the connector intermediate segment 214 and to provide sealing engagement with the O-rings 216, again to restrict the influx of body fluids that could impede electrical performance. The first header bore intermediate section 226 can, in some examples, be provided with a tubular metallic contact 232 that is designed to make electrical contact with the terminal ring contact 220. The tubular contact 232 can be placed in the first header bore intermediate section 226 via an opening 234 that leads from the exterior of the header housing 202 to the intermediate section 226. The opening 234 can be sealed with epoxy, silicone rubber or like adhesives after insertion of the contact 232. An electrical pathway between the tubular contact 232 and the circuitry inside the can 106 may be established by a conductor wire 236 that is connected at one end to the contact 232. The other end of the conductor wire 236 can be fed into the interior of the can 106. In some examples, a biasing member secured within a connector housing can be designed to make electrical contact with the terminal ring contact 220, as shown in FIG. 11.

The header housing 202 can be formed or otherwise provided with a second header bore 238 that can be countersunk and capped by a penetrable septum 240, which can be coupled to the header housing 202. The septum 240 can be secured to the header housing 202 by a suitable biocompatible medical grade adhesive, such as silicone adhesive or like adhesives. The septum 240 can be provided with a slot 242, the function of which is described below.

A connector assembly 250 can be disposed within an injection molding of the header housing 202, adjacent the second header bore 238. The connector assembly 250 can include a rectangular connector housing 252, for example, that is seated beneath the septum 240. The connector housing 252 can have a longitudinal first connector bore 254 that is aligned with the proximal section 230 of the header bore 208 so that the terminal pin contact 222 of the connector 206 is axially received in the connector housing 252. A set-screw 256 can be threadingly connected to the connector housing 252 to retain the terminal pin contact 222 within the housing 252. The slot 242 in the septum 240 can be provided to permit a wrench or other tool to be inserted through the septum 240 to tighten or loosen the set-screw 256, as desired. A biasing member 260 can be disposed in the connector housing 252 and functions to urge the terminal pin contact 222 into contact with a wall of the first connector bore 254, thereby providing secondary retainment (beyond the set-screw 256) of the terminal pin contact 222 within the housing 252.

The connector housing 252 can function as an electrical contact to carry signals to and from the terminal pin contact 222. An electrical pathway between the connector housing 252 and the can 106 may, for example, be established by a conductor wire 262 that is fed at one end into the can 106 and is connected at the other end to the housing 252.

The connector housing 252 and set-screw 256 can be fabricated from a biocompatible metallic material, such as stainless steel, MP35N alloy, titanium or similar materials. The septum 240 can be composed of biocompatible molded plastic, silicone rubber or like materials.

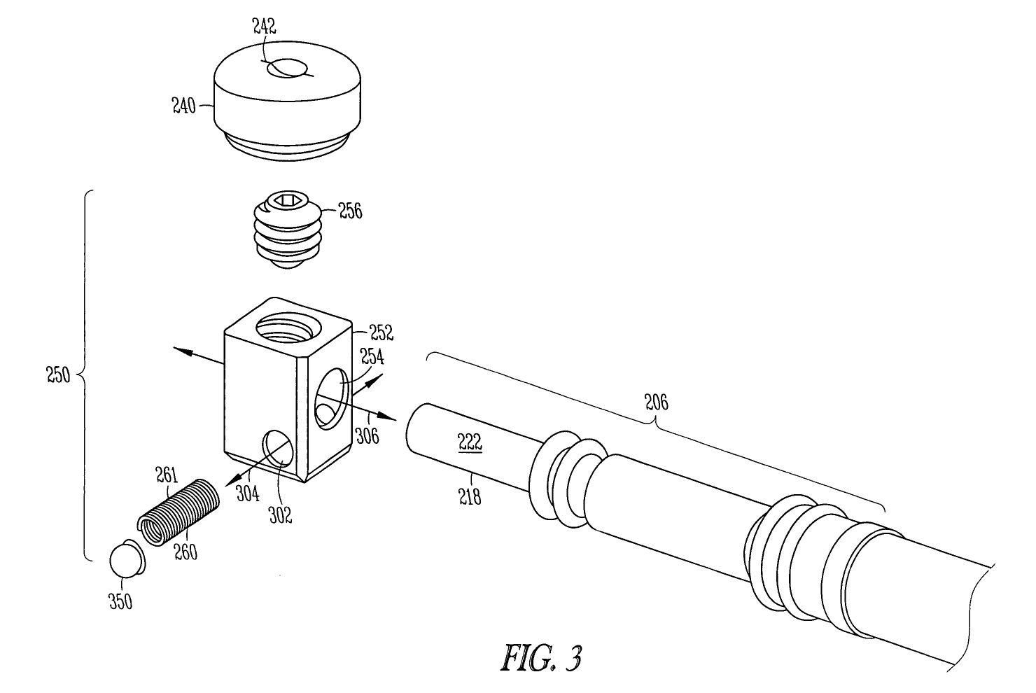

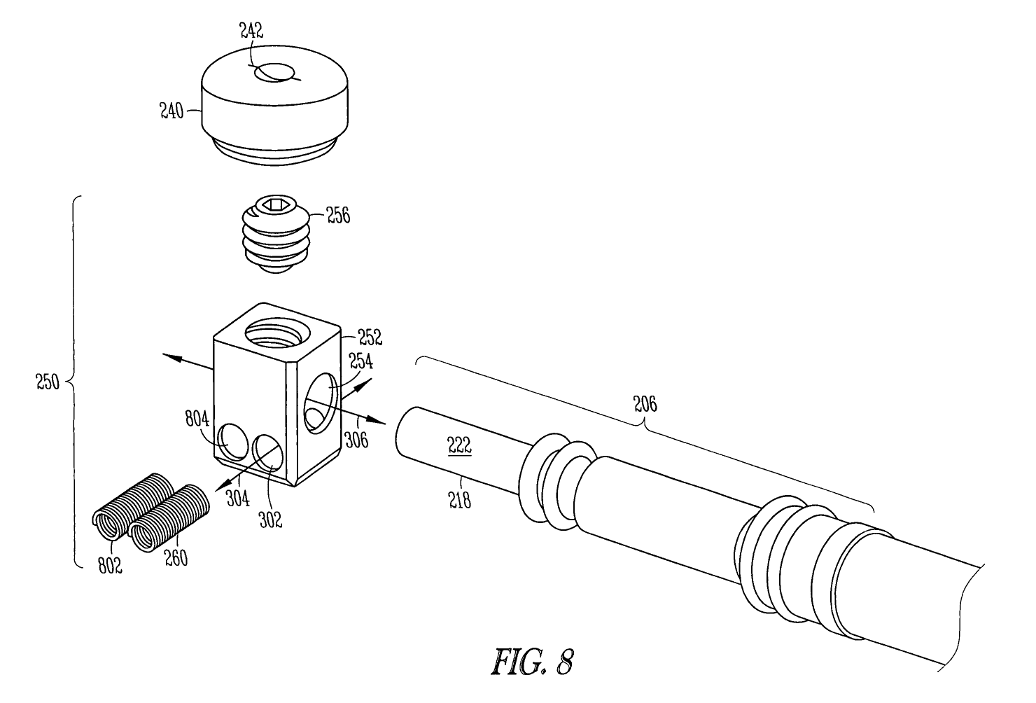

The detailed structure of the connector assembly 250 can be further understood by referring to FIGS. 3, and 5-11, among others. FIG. 3 is an exploded schematic view of the connector assembly 250 and the lead connector 206. FIG. 7 is an unexploded schematic view of the connector assembly 250 with the connector housing 252 and the septum 240 shown in section. As noted above, the first connector bore 254 can be oriented to align with the proximal section 230 of the header bore 208 (FIG. 2) when the connector housing 252 is injection molded within the header housing 202. The biasing member 260 can be disposed in a second connector bore 302 in the connector housing 252 that runs normal or at an oblique angle to the first connector bore 254. The second connector bore 302 can be offset vertically from the first connector bore 254 so that when the biasing member 260 is disposed in the second bore 302, the set-screw 256 may be fully tightened down without interfering with the biasing member 302.

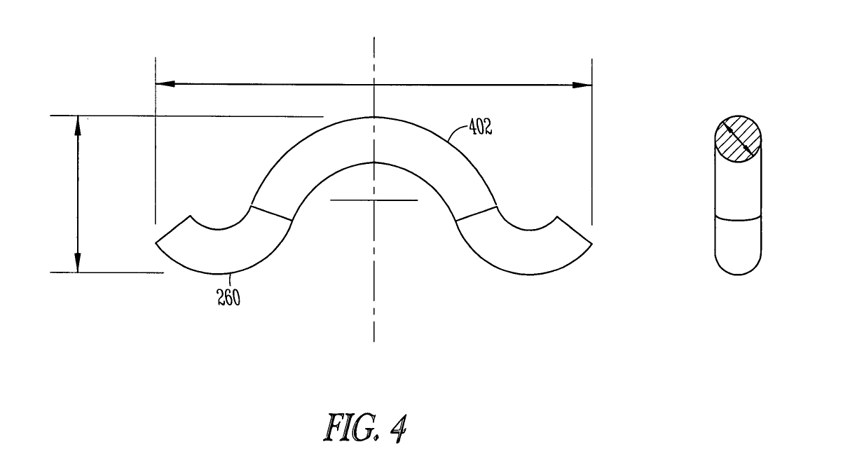

The biasing member 260 can be fabricated in a variety of configurations to provide a desired spring effect. In some examples, such as is shown FIGS. 2-3, the biasing member includes a coiled spring 261 composed of Nitinol, which advantageously resists deformation upon lead connector 206 insertion into and withdrawal out of the connector housing 252. Additional materials that may be used to form the coiled springs can include spring steel, stainless steel, or like biocompatible highly elastic materials. In an example, the coiled spring 261 includes a length of about 0.1 inches and a diameter of about 0.025 inches. In some examples, such as is shown in FIG. 4, the biasing member 260 can be in the form of a bent wire 402. In an example, the bent wire 402 includes a length of about 0.1 inches and a height of about 0.035 inches.

The biasing member 260 can be completely contained within the connector housing 252 via a biasing member cover 350, thereby allowing the connector assembly 250 to be injection molded within the header housing 202 (FIG. 2) and preventing the biasing member 260 from pulling out of the assembly 250 upon lead withdrawal. As shown in exploded view, the biasing member cover 350 can overlie the biasing member 260 and fittingly engage an outer portion of the second housing bore 302 when assembled. The engagement between biasing member cover 350 and the second housing bore 302 inhibits a header housing material (e.g., epoxy, molded plastic or like materials) from seeping into the second connector bore 302 during injection molding of the header housing 202. In some examples, the biasing member cover 350 can abut an annular recessed shoulder formed in the second connector bore 302. In some examples, the biasing member cover 350 includes a diameter of about 0.045 inches, a thickness of about 0.010 inches, or is composed of MP35N alloy, stainless steel or titanium.

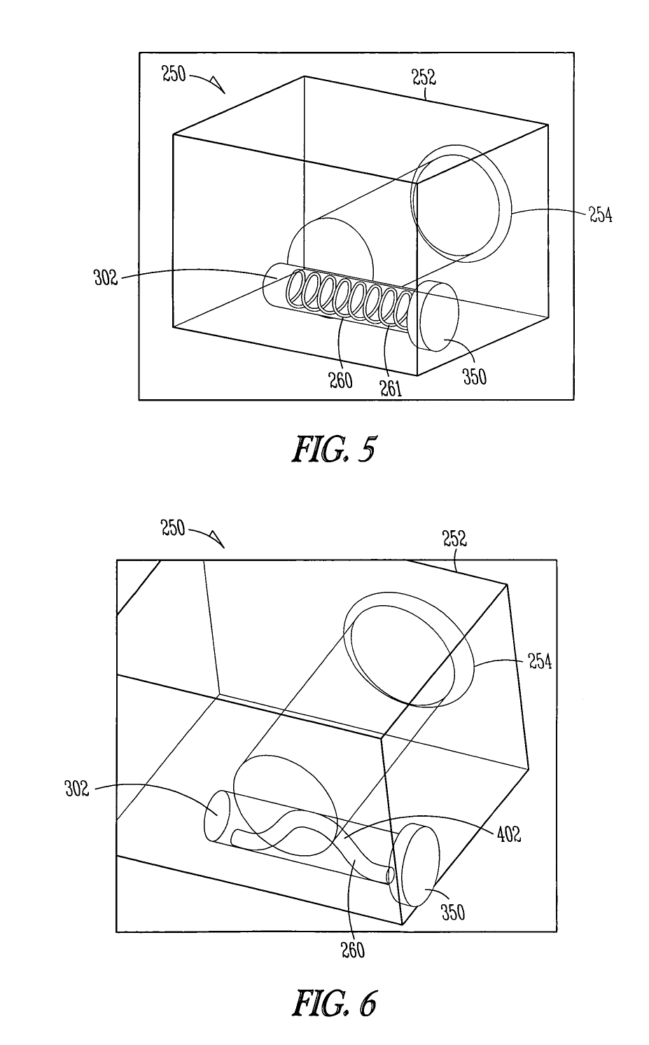

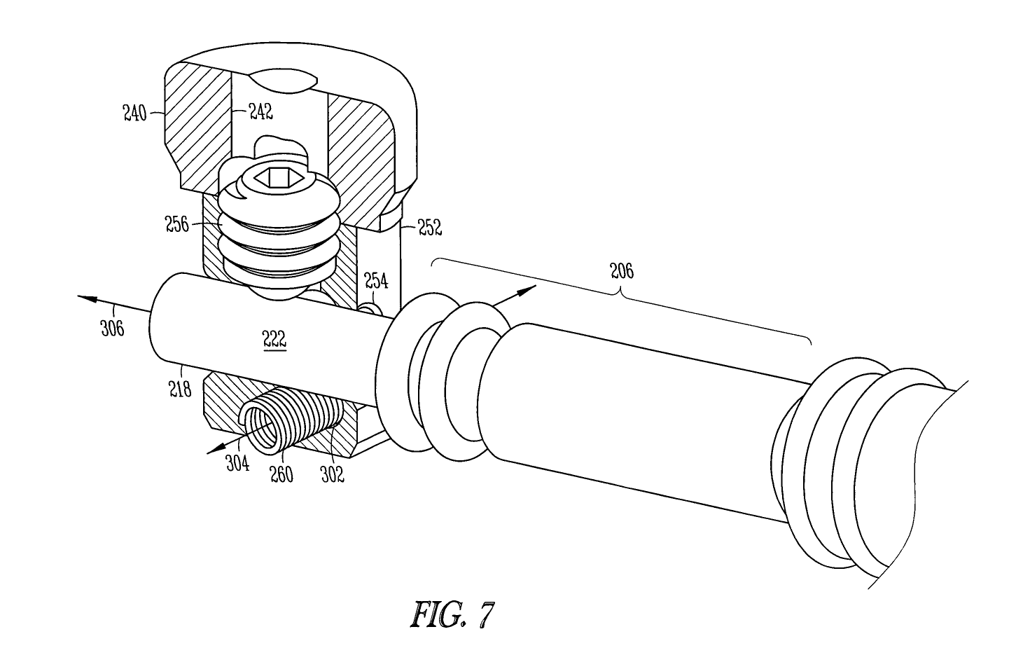

As shown in FIGS. 5-6, the second connector bore 302 and the first connector bore 254 can be relatively disposed so that a portion of the biasing member 260 projects into the first connector bore 254. In this way, the biasing member 260 (e.g., a coiled spring 261 or a bent wire 402) can engage a portion of the connector 206, such as the terminal pin contact 222 or the terminal ring contact 220, when the connector 206 is inserted into the first connector bore 254. In some examples, the longitudinal axis 304 of the second connector bore 302 can be normal to the longitudinal axis 306 of the first connector bore 254, but need not be so long as the relative orientations of the bores 302 and 254 permit a portion of the biasing member 304 to project into the bore 254.

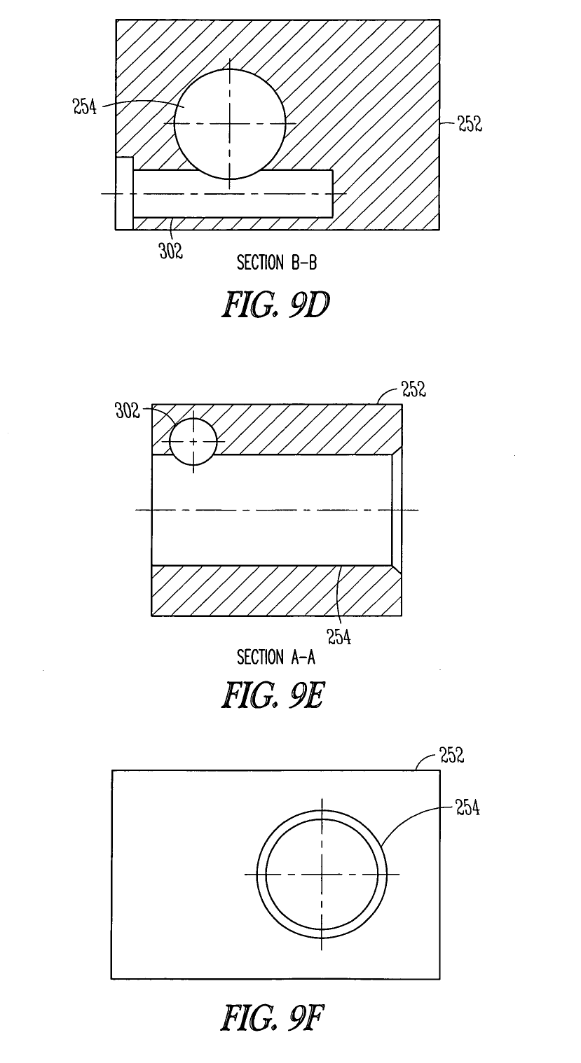

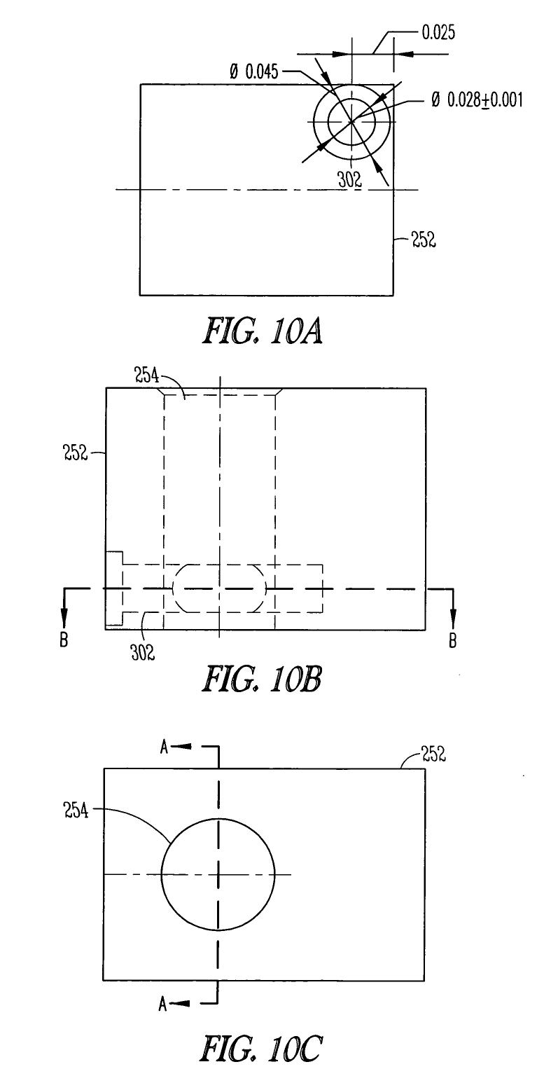

FIGS. 9A-9F and 10A-10F illustrate orientation views of the connector housing 252. In various examples, a center of the second connector bore 302 is disposed at least about 0.025 includes from an edge of the connector housing 252. It has been found that such spacing helps reduce or eliminate the tendency of the header housing material from seeping into the second connector bore 302 during injection molding of the header housing 202. In the example of FIGS. 9A-9F, an interference overlap 902 between the first housing bore 254 and the second housing bore 302 is less than an interference overlap 1002 present in the example of FIGS. 10A-10F.

In the example of FIG. 7, the biasing member 260 biases the proximal segment 218, including the terminal pin contact 222, against the upper wall of the first connector bore 254. Force can be applied to the terminal pin contact 222 by the biasing member 260 in essentially one direction to ensure that the pin 222 is in continuous contact with the walls of the first connector bore 254 immediately upon insertion into the bore 254 and before the set-screw 256 is tightened. As a result, electrical signals can be passed from the cardiac stimulator 102 to the heart as soon as the terminal pin contact 222 is inserted into the first connector bore 254. In addition, the biasing member 260 can resist disconnection of the terminal pin contact 222 from the connector housing 252 in the event the set-screw 256 loosens after implantation.

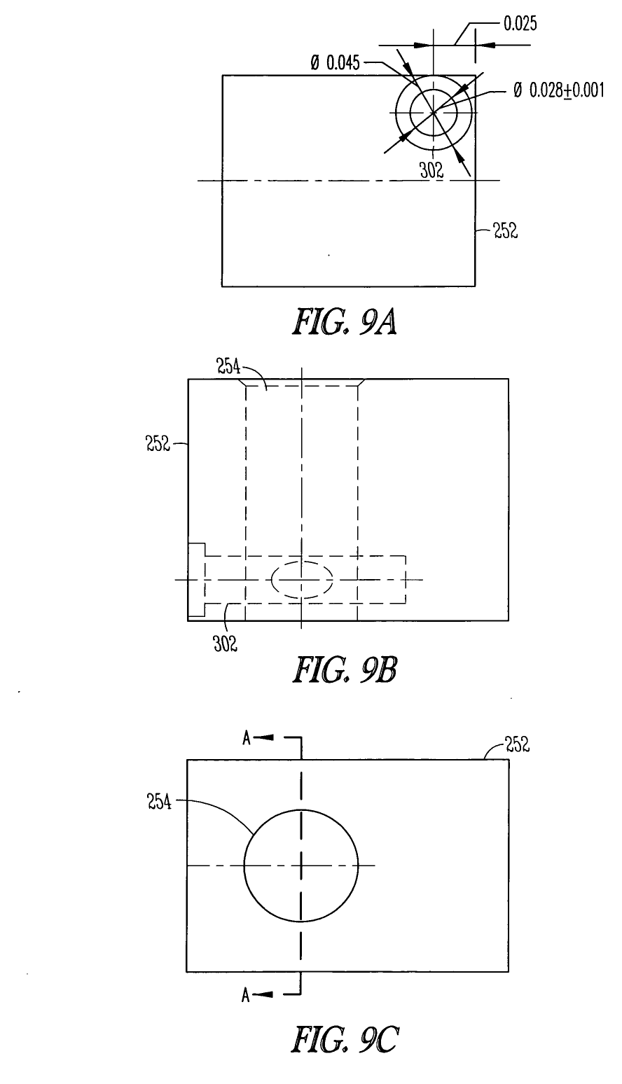

FIG. 8 is an exploded schematic view similar to FIG. 3 and illustrates an another example of the connector assembly 250. FIG. 8 illustrates that the connector assembly 250 can be fitted with at least a second biasing member 802 disposed in a third connector bore 804 in the connector housing 252. Additional biasing members beyond the second biasing member 802 may be provided as needed to contact terminal contacts on the lead connector 206.

FIG. 11 illustrates an example of a connector assembly 1102 configured to be disposed within an injection molding of the header housing 202 (FIG. 2) and contact one or more terminal ring contacts 220 of a cardiac lead connector 206. In this example, the connector 206 includes three terminal ring contacts 220. The connector housing 252 can have a longitudinal first connector bore 254 that is aligned with the proximal section 230 of the header bore 208 (FIG. 2) so that the terminal ring contacts 220 of the connector 206 are axially received in the connector housing 252. One or more biasing members 260 can be disposed in second and third connector bores, for example, and function to urge the terminal ring contacts 220 into contact with a wall of the first connector bore 254, thereby providing electrical contact of the terminal ring contacts 220 within the housing 252.

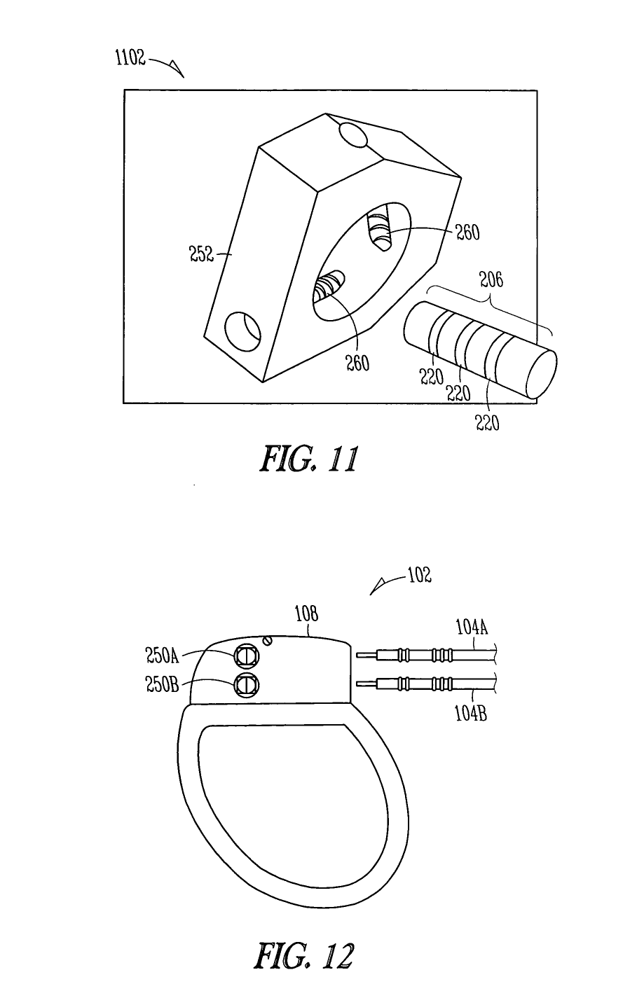

FIG. 12 illustrates a front view of a cardiac stimulator system 100 including a cardiac stimulator 102 and two cardiac leads 104A, 104B for dual-chamber or other dual-site cardiac stimulation. As shown, the header assembly 108 can be provided with two connector assemblies 250A, 250, each of which can be similar in function to the connector assemblies shown in FIG. 3, for example. Additional connector assemblies may be provided to accommodate multiple lead cardiac stimulators.

Among other things, header assemblies for coupling one or more cardiac leads to a cardiac stimulator are described. In varying examples, the header assemblies include a biasing member provided within a connector housing in such a way that it allows the connector assembly to be injection molded into a header housing. The injection molding of the connector assembly within the header housing may advantageously provide for lower cardiac stimulator manufacturing costs, more robust securement of the connector assembly within the header housing and more repeatable alignment between a header bore and a first connector bore, as the connector assembly does not have to be press-fit into the header housing after the header housing has been molded. Further, the header assemblies include a biasing member held within a second connector bore in such a way that the biasing member interferes with the first connector bore, which is configured to receive the proximal end of a cardiac lead. In this way, consistent and immediate electrical contact between the cardiac lead and the connector assembly is possible.

The above Detailed Description includes references to the accompanying drawings, which form a part of the Detailed Description. The drawings show, by way of illustration, specific embodiments in which the invention can be practiced. These embodiments are also referred to herein as “examples.” All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable Inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B.” “B but not A,” and “A and B,” unless otherwise indicated.

In the appended claims, the terms “including” and “in which” are used as the plain-English equivalents of the respective terms “comprising” and “wherein.” Also, in the following claims, the terms “including” and “comprising” are open-ended, that is, a system, assembly, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more features thereof) can be used in combination with each other. Other embodiments can be used, such as by one of ordinary skill in the art upon reviewing the above description. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

The Abstract is provided to comply with 37 C.F.R. § 1.72(b), to allow the reader to quickly ascertain the nature of the technical disclosure. It is submitted with the understanding that it will not be used to interpret or limit the scope or meaning of the claims.

What is claimed is:

1. A header assembly for coupling one or more cardiac leads to a cardiac stimulator, comprising:
a header housing having at least one lead receiving header bore, the header bore having a first longitudinal axis;
a connector assembly injection molded within the header housing, the connector assembly including,
a connector housing having a first connector bore substantially aligned with the first longitudinal axis and a second connector bore;
a biasing member disposed within the second connector bore, the biasing member having a portion projecting into the first connector bore; and
a sealing biasing member cover disposed adjacent an end of the second connector bore; and
a set-screw independent from the biasing member, the set-screw threadingly coupled to a portion of the connector housing and having a lead contact end movable toward or away from the first longitudinal axis.

2. The header assembly of claim 1, wherein the biasing member cover is secured within a portion of the second connector bore, the biasing member cover inhibiting a passage of header housing material into the second connector bore.

3. The header assembly of claim 1, wherein the second connector bore has a second longitudinal axis substantially normal to the first longitudinal axis.

4. The header assembly of claim 1, wherein the second connector bore has a second longitudinal axis disposed at an oblique angle relative to the first longitudinal axis.

5. The header assembly of claim 1, wherein a position of the connector assembly within the header housing urges a received lead terminal pin contact against a wall of the first connector bore.

6. The header assembly of claim 1, wherein a position of the connector assembly within the header housing urges a received lead terminal ring contact against a wall of the first connector bore.

7. The header assembly of claim 1, wherein a center of the second connector bore is disposed at least about 0.025 inches from an edge of the connector housing.

8. The header assembly of claim 1, wherein the biasing member includes a coiled spring comprising Nitinol or spring steel.

9. A header assembly for coupling a cardiac lead having at least first and second terminal ring contacts to a cardiac stimulator, comprising:
a header housing having at least one lead receiving header bore; and
a connector assembly injection molded within the header housing, the connector assembly including,
a connector housing having a first connector bore aligned with the header bore and at least second and third connector bores;
a biasing member disposed within each of the second and third connector bores, each biasing member having a portion projecting into the first connector bore biasing first and second terminal ring contacts of a received cardiac lead against a wall of the first connector bore; and
at least first and second sealing biasing member covers having a size and shape matable with an end of the second and third connector bores.

10. The header assembly of claim 9, further comprising at least one set-screw threadingly disposed in a fourth connector bore and movable toward an axis of the header bore.

11. The header assembly of claim 9, wherein a center of the second and third connector bores is disposed at least about 0.025 inches from an edge of the connector housing.

12. The header assembly of claim 9, wherein at least one biasing member includes a coiled spring comprising Nitinol or spring steel.

13. A method, comprising:
forming a connector assembly, including
forming a connector housing having a lead receiving first connector bore and a second connector bore;
disposing a biasing member within the second connector bore and projecting a portion of the biasing member into the first connector bore; and
sealing an end of the second connector bore and containing the biasing member within the connector housing, including coupling a biasing member cover within a portion of the second connector bore;
injection molding a header housing over the connector assembly, including forming at least one header bore substantially aligned with the first connector bore; and
threadingly coupling a set-screw, independent from the biasing member, to a portion of the connector housing for securing a received cardiac lead to the connector housing when tightened.

14. The method of claim 13, wherein sealing the end of the second connector bore includes inhibiting a header housing material from passing into the second connector bore during injection molding of the header housing.

15. The method of claim 13, wherein disposing the biasing member includes slightly interfering with a circumference of the first connector bore ensuring electrical contact between a proximal end of the received cardiac lead and the connector housing.

16. The method of claim 13, wherein forming the connector housing includes disposing a longitudinal axis of the second connector bore substantially normal to a longitudinal axis of the first connector bore.

17. The method of claim 13, wherein forming the connector housing includes disposing a longitudinal axis of the second connector bore at an oblique angle relative to a longitudinal axis of the first connector bore.

18. The method of claim 13, wherein disposing the biasing member includes disposing a Nitinol or spring steel coiled spring within the second connector bore.

19. The method of claim 13, wherein disposing the biasing member includes biasing a terminal pin contact of the received cardiac lead against a wall of the first connector bore.

20. The method of claim 13, wherein disposing the biasing member includes biasing a terminal ring contact of the received cardiac lead against a wall of the first connector bore.

21. The method of claim 13, wherein forming the connector housing includes forming the second connector bore at least about 0.025 inches from an edge of the connector housing.
